(19) United States
(12) Patent Application Publication
(10) Pub. No.: US 2016/0250065 A1
(43) Pub. Date: Sep. 1, 2016

(54) ELECTRICALLY HEATED BLANKET WITH SLEEP DEPTH -DEPENDENT CONTROL- FOR WEIGHT LOSS

(71) Applicant: Manfred FUHS, Bad Neuenahr (DE)

(72) Inventor: MANFRED FUHS, BAD NEUENAHR (DE)

(21) Appl. No.: 15/029,631

(22) PCT Filed: Jan. 27, 2014
(86) PCT No.: PCT/IB2014/000085

§ 371 Date: Apr. 15, 2016

(30) Foreign Application Priority Data
Oct. 18, 2013 (DE) 20 2013 009 227.0

(51) Int. Cl. A61F 7/00; H05B 1/02; H05B 3/56; A61B 5/00; H05B 3/34

(52) U.S. Cl. CPC A61F 7/007; A61F 7/0097; A61B 5/4812; H05B 3/342; H05B 3/56; H05B 1/0272; H05B 1/0202; A61F 2007/029

(57) ABSTRACT

An electric blanket is controlled so that it initially provides cozy warmth at low ambient temperature, but on reaching deep sleep phases, temperature is lowered, for as much dissipation of calories as it does not disrupt the sleep.

### FIELD OF THE INVENTION

The invention relates to a bed cover, more particularly to an electric blanket, which, however barely isolates, but only temporarily produces electrical warming.

### BACKGROUND OF THE INVENTION

### Problem to be Solved

The known physiological effect that light clothing and some shivering increase calorie consumption and thus helps in weight loss, has already led to many proposed concepts in diet, light clothing and to strive for more physical action.

However, the physiological effects causing chills with small, involuntary contractions of subcutaneous muscles is discomforting to most obesed persons and is therefore avoided.

Additionally, there is the old superstition that colds were caused by temporal hypothermia—even though catching a cold is known to be caused by infections.

Therefore, such recipes have rarely been complied with.

### Prior Art

A technological solution to this problem is suggested in patent applications of Li Jun in CN2870640 (Y) Deshui Li with CN201888580 (U), as well as Hanschke in DE 10 2010 033 971 A1, which propose a blanket with circulating coolant, while several other innovations suggest the injection of cooling (or, for other purposes warming) air or transfer agents into bed covers, as proposed in DE 692 12 211 T2.

It is important to note however, that blankets with circulation of coolants inevitably must be rather stiff and uncomfortable and would be severely impaired in their function by inevitable kinks.

Moreover, the system requires high technical complexity for waterproofing or airtightness and maintaining circulation.

In addition, application is bound to high self-discipline, since the blankets already spread chill before falling asleep.

### Object of the Invention

It is therefore an object of the present invention, to develop a system for high user compliance.

This is achieved with only chilling the body within deep sleep phases and with a design, that is inexpensive and less intricate to use.

### Inventive Step

The inventive step therefore is to control the heating with an analysis of the state of sleep, which is expediently done by inserting sensors into the blanket and to refrain from any active cooling, but to design the blanket so that it isolates the body heat as little as possible in its passive state and rises temperature only through the electric heating function which can easily be controlled.

### Techno-Medical Basis

The sleep cycle comprises a change from deep sleep to light sleep with brief almost-awake moments. In this phase, possible cooling is perceived and would lead to awakening.

The average adult has about 4-5 full sleep cycles within 8 hours. Each cycle lasts between 90 and 110 minutes, and has 5 different phases, which vary individually and on external influences.

Phase 1 marks falling asleep, in phases 2 to 4 of the sleep grows deeper for the so-called delta sleep status, during which cooling causes no reduction in the depth of sleep or would provoke waking up.

The 5th Phase is called REM phase. This is the dream phase of accelerated respiration, increased brain activity and rapid eye movements under the eyelid (hence REM is the acronym for Rapid Eye Movement).

At this stage cooling would result in impairment of the sleep quality and therefore has to be avoided.

With the aid of an acceleration sensors, the short light-sleep phases (particularly the critical REM-phases) can be determined and largely predicted by the sequence analysis, so that the blanket can be heated up again before such a critical moment occurs.

### DESCRIPTION OF THE INVENTION

The blanket consists of a heating fabric as in conventional electric blankets, only that it is covered on both sides with lattice or heat-permeable fabric.

A few three-dimensional position sensors, in a one-chip design are integrated and report to an analytic electronic device.

The occurrence of position changes, which are registered this way, build the parameters for the identification of sleep phases, which may be assigned as to common model and on this basis be continuously predicted, however improved with increasing self-learning of the system on basis of evolution of registered data.

In deep sleep phases, normally characterized in a lack of movement—but fine tuned by appropriately corrected analysis—the heating of the blanket is reduced or turned off. This results in a cooling effect even at ambient temperatures up to 25° C., which otherwise—though in half-sleep—would cause a user to re-arrange his blanket.

If the user wakes up due to discomfort, he or she can press the sleep button on a small control unit, so that the blanket warms again.

With feeding back equivalent parameters into the control system, switching points and -times can be automatically be fine tuned.

Furthermore, an increased movement of the blanket is sufficient as an indicator that the deep sleep phase is over and the temperature needs to be raised again.

### DESCRIPTION OF THE DRAWINGS

FIG. 1 shows the structure of the blanket 1 with the heating fabric 2, the covering lattice 3 and 4 and the incorporated sensors 5 and 6.

FIG. 2 is a typical diagram that demonstrates the depth of sleep (Y) on the the perpendicular axis in phases from sleep-in and half-sleep 1 up to 4, the deep “delta-phase” on 8 hours sleep time (see clock faces below), until wake-up time 6.

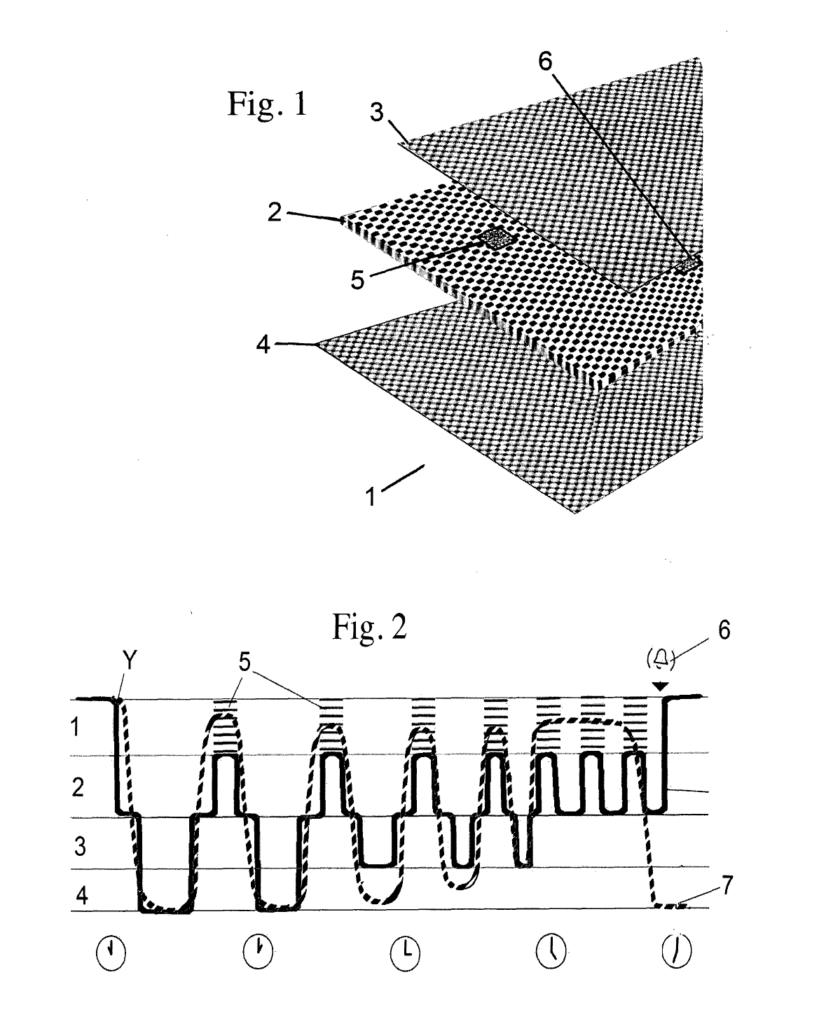

The broken line 7 indicates the heating intensity which needs to be adapted to the sleep phases and can be used for waking up the user by switching off the heating at reveille time.

What is claimed is:

1. Electrically heated blanket with sleep depth-dependent control for weight loss, wherein the blanket has no significant thermal insulation effect.

2. Electrically heated blanket as to claim 1, wherein the heating is controlled in response to sleep phases.

3. Electrically heated blanket as to claim 1, wherein the the sleep phases are determined by motion sensors in the blanket, connected to an recording logic.

4. Electrically heated blanket as to claim 3 wherein coming sleep phases are predicted by evaluation of registered prior devolutions.

5. Electrically heated blanket as to claim 1, wherein renewed pressure on the start button prior to passage of an ordinary sweep is registered as incident due to possible temperature undercut.

6. Electrically heated blanket as to one or more of the preceding claims, wherein the switch-off of the heating is also used for or at least supporting waking up of the user at reveille time.
